(19) United States
(12) Patent Application Publication
(10) Pub. No.: US 2018/0122499 A1
(43) Pub. Date: May 3, 2018

(54) COMPUTER-ASSISTED MEDICAL INFORMATION ANALYSIS

(71) Applicant: 3M INNOVATIVE PROPERTIES COMPANY, St. Paul, MN (US)

(72) Inventors: Steven M. Austin, Spring Branch, TX (US); Garri L. Garrison, Leitchfield, KY (US); Khalod S. Kelantan Pelegrin, Park City, UT (US); Jeremy M. Zasowski, Cambridge, MA (US)

(21) Appl. No.: 15/570,795

(22) PCT Filed: Apr. 29, 2016
(86) PCT No.: PCT/US2016/029927

§ 371 Date: Oct. 31, 2017

Related U.S. Application Data
(60) Provisional application No. 62/306,141, filed on Mar. 10, 2016.
(60) Provisional application No. 62/156,702, filed on May 4, 2015.

(51) Int. Cl. G16H 10/60; G16H 50/30; G16H 50/20; G06F 17/30

(52) U.S. Cl. CPC G16H 10/60; G06F 17/30371; G16H 50/20; G16H 50/30

(57) ABSTRACT

In general, systems and techniques for managing and updating medical information are described. In one example, a computer-implemented method for managing medical information is described. The method includes receiving current diagnostic information that includes one or more current individual diagnoses for a patient, comparing the current diagnostic information to prior diagnostic information that includes one or more prior individual diagnoses for the patient, determining one or more possible errors in the current diagnostic information based on the comparison, and generating an alert associated with each possible error of the one or more possible errors.

This application is related to U.S. Provisional Application No.: 62/156,702, filed on May 4, 2015, and U.S. Provisional Application No.: 62/306,141, filed on Mar. 10, 2016, the entire content of which are incorporated by reference herein.

### TECHNICAL FIELD

This disclosure relates to systems and techniques for managing and updating medical information.

### BACKGROUND

In the medical field, accurate processing of records relating to patient visits to hospitals and clinics ensures that the records contain reliable and up-to-date information for future reference. Additionally, various external entities, such as insurance providers, rely on receiving up-to-date information from hospitals and clinics to accurately perform their respective functions. Accurate processing is also useful for medical systems and professionals to receive prompt and precise reimbursements from insurers and other payers.

### SUMMARY

In general, this disclosure describes systems and techniques that flag possible errors and/or omissions of medical information, such as with respect to erroneous data entry in medical documentation or records. Various medical staff include physicians, clinicians (e.g., physicians' assistants, nurses, other healthcare professionals, etc.), and medical coders. One or more medical staff may document medical information for a given patient. In turn, insurers and/or other payers may access the medical information, and generate various data from the collected medical information. For example, the medical staff may document diagnoses and various medical conditions for a patient, a medical coder may record the diagnoses and conditions as codes, and an insurer may generate a risk level for the patient from the recorded codes. By assigning a risk level to a patient, the insurer and other payers may more accurately determine funding amounts and other monetary and non-monetary parameters associated with the patient's insurance.

Aspects of this disclosure are directed to flagging possible errors and omissions (collectively referred to as “errors” herein) to various staff in the medical field, such as payers, physicians, clinicians, and medical coders. For example, systems and techniques described herein may analyze diagnoses and medical conditions entered by medical staff, and alert the staff/payers to possible errors in the entered information. Oversights and inadvertent errors committed by medical staff while entering the information can potentially harm the accuracy of risk level calculations, which are performed by insurers and payers using the most recently entered information. To mitigate or circumvent these potential inaccuracies, systems and techniques of this disclosure analyze the information entered by medical staff for possible errors, and provide prompts and suggestions to the medical staff to rectify the detected potential errors.

In one example, this disclosure describes a computer-implemented method for managing medical information. The method may include receiving, by a computing device, current diagnostic information for a patient, the current diagnostic information including one or more current individual diagnoses for the patient; comparing, by the computing device, the current diagnostic information to prior diagnostic information for the patient, the prior diagnostic information including one or more prior individual diagnoses for the patient; determining, by the computing device, one or more possible errors in the current diagnostic information based on the comparison; and generating, by the computing device, an alert associated with each possible error of the one or more possible errors.

In another example, this disclosure describes a computer-implemented method for managing medical information, the method including receiving, by a computing device, an alert indicating a possible error in current diagnostic information for a patient as compared to prior diagnostic information for the patient; and outputting, by the computing device, a prompt requesting correction of the possible error.

The systems and techniques described herein provide several potential advantages. For instance, by flagging possible errors in entered medical codes, aspects of this disclosure may enable medical staff to provide a patient with a more accurate assessment of the patient's health information. In turn, the patient may be able to make more informed decisions with respect to healthcare and lifestyle. Additionally, the error-detection systems of this disclosure may enable medical staff to provide a more accurate depiction of a patient's medical conditions to the insurers and payers, thus improving the accuracy of risk level assignments and other insurance-related determinations. In addition, by flagging such possible errors for multiple staff members (e.g. payers, doctors, clinicians, and coders), aspects of this disclosure may enable staff members and/or payers to detect and rectify errors committed by others in the chain of data entry. The error-detection aspects of this disclosure provide the technical benefit of systems that are better equipped to trap data errors, to enable greater error resilience, and to mitigate potential propagation of data errors.

The details of one or more examples of the described systems, devices, and techniques are set forth in the accompanying drawings and the description below. Other features, objects, and advantages will be apparent from the description and drawings, and from the claims.

### BRIEF DESCRIPTION OF DRAWINGS

FIG. 1 is a block diagram illustrating an example distributed system configured to determine one or more discrepancies within medical information of a patient consistent with this disclosure.

FIG. 2 is a block diagram illustrating the server of the example of FIG. 1.

FIG. 3 is a conceptual diagram illustrating a general schematic associated with the techniques of this disclosure.

FIG. 4 is a block diagram that illustrates a legend for various flowcharts in the accompanying drawings.

FIG. 5 is a flowchart illustrating an example workflow by which physician or a care team member interacts with patients.

FIG. 6 is a screenshot illustrating a user interface (UI) through which physicians can view the current and historical HCC status of a patient, using the client computing device of FIG. 1.

FIG. 7 is a flowchart illustrating a workflow associated with clinical documentation improvement (CDI) specialists' interaction with tools of this disclosure.

FIG. 8 is a screenshot illustrating a query to a physician originating from the CDI specialist.

FIG. 9 is a flowchart illustrating a workflow associated with a community case manager's role in the medical reporting process.

FIG. 10 is a flowchart illustrating a workflow associated with the role of HIM coders, who may verify ICD-9 or ICD-10 diagnoses and related information in a medical record.

FIG. 11 is a flowchart illustrating a workflow for business management personnel and/or analysts who may use analytics for resource planning and contract negotiations.

FIG. 12 is a flowchart illustrating a workflow associated with system analysts who provide HCC data to payers and Medicare as needed, as well as to hospital finance teams that can identify cases subject to internal audit for lack of documentation of existing chronic diseases.

FIG. 13 is a flowchart illustrating an example process that a computing device may perform to implement one or more of the medical information management and updating techniques of this disclosure.

FIG. 14 is a flowchart illustrating an example process that a computing device may perform to implement one or more of the medical information management and updating techniques of this disclosure.

### DETAILED DESCRIPTION

This disclosure describes systems and techniques for detecting possible data entry errors by medical staff, and prompting the medical staff member(s) to rectify the errors. The systems and techniques may be used to flag the potential errors to the medical staff before the entered data is made available to insurers, payers, and other third parties that may use the data in their own decision-making processes. Thus, aspects of this disclosure may prevent patients, insurers, and payers from using erroneous or incomplete data to generate data that can significant influence the patient's health-related decisions and costs.

When medical staff visit(s) with a patient, the medical staff may perform a new examination of the patient, review medical history of the patient, and/or combine the exam results and past history to formulate a holistic medical evaluation of the patient. By entering medical condition data points obtained from new exam results as well as from medical history information, the medical staff may determine (or enable determinations of) coexisting medical conditions, including so-called “co-morbidities.” Synergistic effects of co-morbidities may inform decisions relating to future healthcare steps and insurance funding. Even in cases where co-morbidities are not known to show significant synergistic effects, any updates to the patient's current set of medical decisions may be instrumental in informing healthcare and insurance funding decisions.

To record medical conditions pertaining to a patient, medical staff may enter information that maps to one or more corresponding medical conditions. To facilitate the sharing of medical condition information with third parties, medical staff may enter information that conforms to a standard. For instance, different entities, such as hospitals, clinics, medical transcription agencies, insurers, or payers, may use the data entered by the medical staff in order to inform decisions on healthcare, funding, and other related matters. To that end, standard-setting organizations may formulate one or more standards that dictate particular information and scenarios in which the information is applicable.

Examples of such standards are the International Classification of Diseases (“ICD”) standards. The ninth and tenth revisions of the ICD standards (“ICD-9” and “ICD-10,” respectively) specify numerous codes that identify particular medical conditions. As one example, ICD-9 specifies the code “250.02” for the medical condition “diabetes mellitus without mention of complication, type II or unspecified type, uncontrolled.” Thus, if a medical coder is called upon to record that a patient has the condition of “diabetes mellitus without mention of complication, type II or unspecified type, uncontrolled,” the medical coder would select the code “250.02.” Other coding standards, such as SNOWMED, may be used to code medical information and the disclosure is not limited in this respect.

Subsequently, various third parties may access the ICD-9 or IC-10 (collectively referred to as “ICD”) codes entered by the medical staff, in order to inform various decision-making processes. For instance, an insurer, such as a private health insurance company that provides Medicare (government-administered health insurance) benefits, or a government entity that pays premiums to the insurer, may access the entered ICD codes for a patient, to determine payment and billing information. Aspects of this disclosure are described with respect to Medicare Advantage plans, which are private health plans by which an insurer provides Medicare benefits. However, it will be appreciated that the systems and techniques of this disclosure may be applicable to other types of insurers as well, such as various insurance exchanges that may operate under the auspices of the Affordable Care Act.

A Medicare Advantage plan provider may use the ICD codes available for a patient to project or approximate the patient's health-related risk level. In many instances, Medicare Advantage plan providers assign a patient a risk level that corresponds to a predetermined category in a graded or graduated system. A specific example of such a graded system is the Hierarchical Condition Categories (or “HCC”) system created and maintained by the Center for Medicare & Medicaid Services (CMS). CMS is an agency within the Department of U.S. Health and Human Services.

The risk level assignment system using HCC grades was created for the purpose of implementing a risk-adjusted, prospective payment scheme based on the relative cost of care for healthy populations versus sick populations. HCC grades (hereinafter, “HCCs” were first developed specifically for Medicare Advantage plans in 2004. As discussed above, Medicare Advantage plans are health plans that Medicare-eligible individuals join and are administered by private, third party insurers. For payers and providers, Medicare Advantage plans feature a prospective payment system, typically based on a Per-Member-Per-Month (PMPM) rate. HCCs play a large part in determining the PMPM rate, and payers and providers share in the cost risk (and savings) of providing care to the population.

An HCC includes a numeric code and corresponding description that indicates a patient's health status as supported by current diagnoses from one or more medical staff members (i.e., physicians). Each individual diagnosis is recorded using an ICD-9 or ICD-10 code. In turn, the ICD-9 and/or ICD-10 codes are mapped to an HCC number. Using the example above, the ICD-9 code 250.02 (with the description “diabetes mellitus without mention of complication, type II or unspecified type, uncontrolled”) maps to the following HCC: “19—Diabetes without complications.” As shown, the numeric component of the corresponding HCC is “19” and the descriptive component is “diabetes without complications.”

The HCC-to-ICD mapping is a many-to-one relation at present. Currently, approximately 3,300 ICD-9 diagnosis codes are mapped to 187 HCCs. Patients can, and often do, have multiple HCC-affecting medical conditions at the same time. In some instances, these multiple medical conditions are recorded using multiple ICD codes. In turn, a patient's HCC is determined cumulatively, based on all of the patient's current medical conditions, as indicated by the latest recorded ICD codes. Each HCC has an associated “risk adjustment factor” represented by a fractional number above or below 1.0. Greater numbers of the risk adjustment factor are associated with higher payments, based on a greater risk level of a patient who has more health issues than average. Conversely, a lesser number for the risk adjustment factor represents a healthier patient, which is associated with a lesser risk level.

The distribution of HCCs in a patient population is a contributing factor in reimbursement arrangements between Medicare Advantage providers and payers (e.g., CMS), as well as between payers and providers. HCCs, risk factors, and payment rates for a given patient are adjusted once every calendar year. As discussed above, a Medicare Advantage provider receives a PMPM payment rate for a given patient from a payer, such as CMS. The PMPM rate for a particular patient is based on the cumulative, adjusted HCC assigned to the patient, and not on specific diagnoses indicated by the corresponding ICD codes. Different HCCs are associated with different PMPM payment rates.

The existing systems described above present several potential issues with respect to implementation. Generally, there are five challenges that the HCC system poses to healthcare providers, patients, insurers, and payers in the context of Medicare Advantage plans. Aspects of this disclosure address, by mitigating or eliminating, administrative challenges and revenue risks associated with Medicare Advantage plans that are run based on the HCC system. A first potential challenge is that diagnoses must be documented correctly in the medical record, especially when dealing with co-morbidities. A second potential challenge is that diagnoses must be updated and documented annually, based on an in-person (face-to-face) evaluation of the patient by a physician. In other words, diagnoses must be reassessed and documented each year from an in-person evaluation by a provider (e.g., physician).

A third potential challenge presented by the existing HCC system is that the system needs to include population management methodologies with the ability to link together a patient's multiple encounters (e.g., visits with medical staff) within the network. A fourth potential challenge is that management of HCCs requires the coordination of multiple levels of activity and services within the network (e.g., physicians, clinicians, administrative staff, information technology workers, and so on). A fifth potential challenge is that effective HCC management requires an analytics model that accurately portrays historical, current, and projected HCC data.

Aspects of this disclosure may address these potential challenges by integrating calculation and awareness of a patient's HCC status as well as an entire population's HCC experience throughout the workflows of the practitioners who access and sometimes influence the HCC score. As discussed above, the HCC system forms an established reimbursement methodology used for Medicare Advantage plans, and many commercial healthcare providers have adopted the HCC-based methodology for capitated payment programs (e.g. Kaiser Permanente). Techniques and systems of this disclosure may enable customers (e.g., healthcare providers or insurers) to more effectively and more accurately manage their patient population, leading to more accurate reimbursement and contract negotiations.

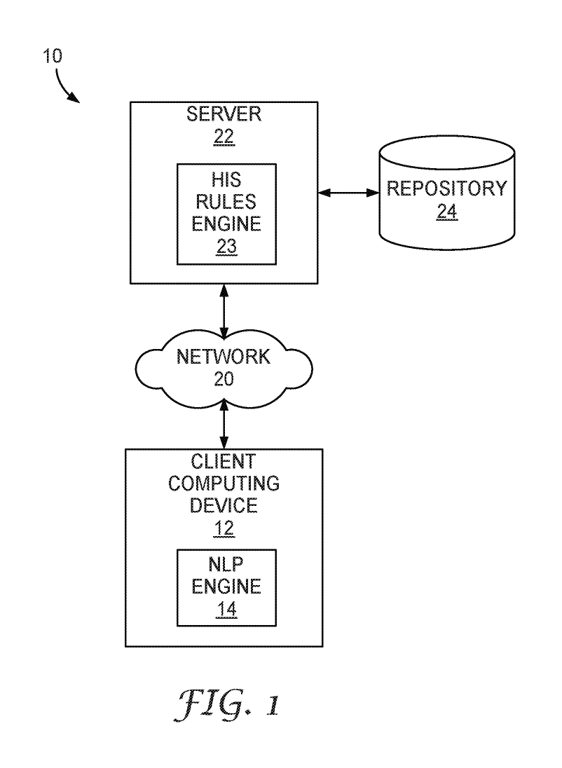

FIG. 1 is a block diagram illustrating an example distributed system configured to determine one or more discrepancies within medical information of a patient consistent with this disclosure. As described herein, system 10 may include one or more client computing devices 12, a network 20, server computing device 22, and repository 24. Client computing device 12 may be configured to communicate with server 22 via network 20. Server 22 may receive various requests from client computing device 12 and retrieve various information from repository 24 to address requests from client computing device 12. In some examples, server 22 may generate information, such as suggested codes and queries for client computing device 12.

Server 22 may include one or more computing devices connected to client computing device 12 via network 20. Server 22 may perform the techniques described herein, and a user may interact with system 10 via client computing device 12. Network 20 may include a proprietary or non-proprietary network for packet-based communication. In one example, network 20 may include the Internet, in which case each of client computing device 12 and server 22 may include communication interfaces for communicating data according to transmission control protocol/internet protocol (TCP/IP), user datagram protocol (UDP), or the like. More generally, however, network 20 may include any type of communication network, and may support wired communication, wireless communication, fiber optic communication, satellite communication, or any type of techniques for transferring data between two or more computing devices (e.g., server 22 and client computing device 12).

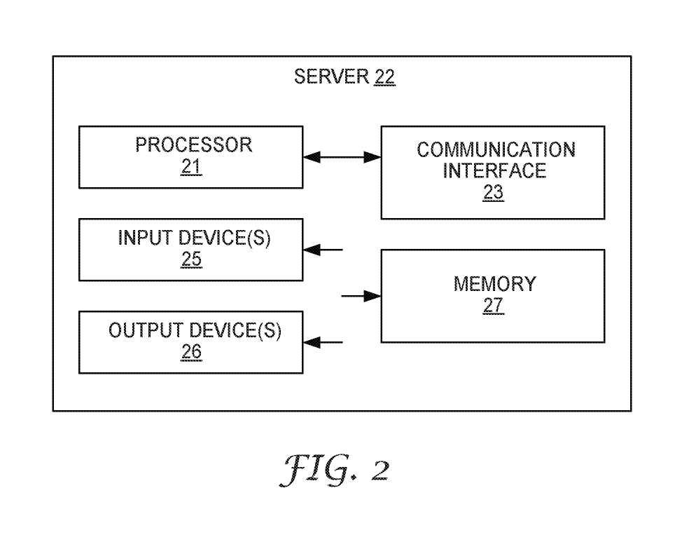

Server 22 may include one or more processors, storage devices, input and output devices, and communication interfaces as described in FIG. 2. Server 22 may be configured to provide a service to one or more clients, such as determining discrepancies within medical information (e.g., between different types of medical information), generating and outputting queries that identify discrepancies to the physician, and resolve the discrepancies based on additional user input. Server 22 may operate on within a local network or be hosted in a Cloud computing environment. Client computing device 12 may be a computing device associated with an entity (e.g., a hospital, clinic, university, or other healthcare organization) that provides information to a physician during a patient encounter and/or receives input documenting aspects of the patient encounter. Examples of client computing device 12 include personal computing devices, computers, servers, mobile devices, smart phones, tablet computing devices, etc. Client computing device 12 may be configured to upload generated medical information to server 22 for analysis and determination of any discrepancies by server 22. Alternatively, client computing device 12 may be configured to retrieve queries and/or other information generated by server 22 and stored in repository 24. Server 22 may also be configured to communicate with multiple client computing devices 12 associated with the same entity and/or different entities.

When a physician sees a patient in either an outpatient clinic or during an office visit (e.g., a patient encounter), the physician typically performs an evaluation of the patient, the patient's medical history and/or the patient's current medical condition. The physician may also perform a medical procedure on the patient during the patient encounter or prescribe treatment related to the patient's medical condition.

In order to be reimbursed for these medical services, a physician may submit, via client computing device 12, a claim for the services performed during the patient encounter. In some examples, such as in inpatient settings, the physician may enter descriptions of one or more of diagnoses, conditions, symptoms, or procedures that are recommended that were or will be performed. In some examples, such as in outpatient settings, the physician may also submit diagnostic information using appropriate diagnosis codes (e.g., ICD-9 or ICD-10 codes) related to the patient's condition(s).

In various instances, other medical staff, such as clinicians, may enter the diagnosis codes using client computing device 12. In still other instances, medical coders may enter the diagnosis codes using client computing device. Medical coders may be referred to herein as health information management or “HIM” coders. As shown, various medical staff may perform the actual entering of diagnosis codes, such as ICD codes for a patient's condition(s), based on the most recent patient examination by the physician. Thus, ICD codes for a patient can be entered at client computing device 12 during various stages of the medical evaluation and reporting processes. For instance, the clinicians and/or HIM coders may review verbose descriptions recorded by the physician, and enter the corresponding ICD codes.

In turn, the insurer, such as a Medicare Advantage plan provider, may access the ICD codes from server 22 (which, in turn, may store the entered ICD codes to repository 24). Insurers may access the ICD codes from server 22 from various remote locations, using their respective client devices (not shown in FIG. 1). As discussed above, the insurer may use the ICD codes accessed from server 22 to generate an HCC level for the corresponding patient.

One or more components of system 10 (e.g., client computing device 22) may implement techniques of this disclosure to provide an integrated, automated, and relatively comprehensive approach for healthcare provider networks to assign and manage HCCs within their covered member and patient population. System 10 may implement processes of this disclosure to make a direct impact on the organization's reimbursement and ability to manage financial risk in an HCC-based capitated payment environment.

Several aspects of the disclosed techniques are described below. A first aspect involves Natural Language Processing. One or more components of system 10 may implement a natural language processing (NLP) engine, such as NLP engine 14 of client computing device 12. NLP engine 14 may extract required patient demographic data elements or risk factors (e.g., age, gender, disability status, original reason for entitlement, Medicaid eligibility, and other elements are listed and explained in 70.2.3—Demographic Factors in the CMS-HCC Model (CMS Medicare Managed Care Manual)) directly from the medical records accessible via client computing device 14. NLP engine 14 may implement a combination of using structured and unstructured data. For example, client computing device 12 may discern a patient's age from data that populates standard health level 7 (HL7) demographic field. However, client device 12 may use NLP engine 14 to extract the patient's disabled status, Medicaid eligibility, and other factors from relevant documentation documentation or via structured data fields entered by a clinician or staff member via an application (such as 3M® 360 Encompass).

Additionally, server 22 may implement techniques of this disclosure to store longitudinal patient records. As used herein, a longitudinal patient record integrates conditions that instigated the patient encounter, as well as other existing conditions (e.g., chronic conditions) that may be related to the current diagnoses. For instance, server 22 may link evidence for various required elements, such as institutional status, frailty, aged status, and Medicaid status in a longitudinal patient record stored to repository 24. NLP engine 14 may capture some or all of the listed elements from searchable records and documentation. Server 22 may store the captured elements to repository 24 as part of the longitudinal patient record and update the data, if needed, with each patient encounter. Medical staff using client computing device 12 may provide these data points and elements of the longitudinal record through structured and unstructured data feeds (e.g., via 3M® 360 Encompass). Additionally, client computing device 12 and server 22 may use cloud computing solutions to store the elements as part of a longitudinal record (e.g. a 3M® Enterprise Patient Record Store). Using such cloud computing resources, components of system 10 may make the longitudinal records available to various entities that may need to access the elements of the record.

Health information system (HIS) rules engine 23 of server 22 may link conditions and diagnosis codes (ICD9 or ICD10) with corresponding HCCs. In turn, server 22 implements one or more techniques of this disclosure to detect possible errors or omissions in the ICD codes. Server 22 may also push queries and/or alerts to client computing device 12, to prompt medical staff members to rectify the possible errors/omissions. Sever 22 may identify situations where greater specificity is needed in the documentation provided by the physician (or other medical staff), to assign the HCC accurately. For instance, server 22 may push these alerts to client computing device 12 to be presented (or “surfaced”) to the physician as automated queries, as well as to clinicians and/or clinical documentation specialists, HIM coders, etc. as the case may be.

In various use cases, certain conditions may trigger server 22 to generate suggestions for medical staff members to examine or reexamine at the language of the diagnosis, or to look for and document other co-morbidities that could lead to a more accurate HCC risk score. For instance, “diabetes with peripheral vascular disease manifestations” may trigger a different HCC risk score than “diabetes without complications.” Client computing device 12 may output these prompts in the form of a query from a clinical documentation improvement (CDI) specialist to a physician, or in the form of an automated prompt/query directly to the physician.

To generate alerts to be pushed to client computing device 12, server 22 may implement the techniques to incorporate public domain information, CMS defined information, and HCC risk scoring methodology to calculate and update HCC risk level assignment. Additionally, NLP engine 14 may capture, and server 22 may store to repository 24, HCC scoring and status of a patient within the patient's longitudinal record. Server 22 may expand the patient record, such as an Enhanced Patient Record System (EPRS) record definition to include relevant HCC data. Additionally, server 22 may link HCC information to documents and claims from all encounters within the healthcare network, as captured in the longitudinal record. Today, computer assisted coding solutions (CAC) solutions, such as those provided by 3M®, are capable of linking a suggested code to the phrases and terms within the documentation as “evidence” of the suggested code. CAC solutions (implemented via one or more devices of system 10) may link suggested codes in this way by marking up certain versions of the corresponding document. Server 22 may implement a similar approach to “mark-up” the documents for HCC data. By implementing techniques of this disclosure, server 22 may expand HCC mark-up data (which are generally diagnosis codes), to include the demographic data as well. For instance, server 22 may generate the HCC data for a patient, according to techniques of this disclosure, to include a demographic component (e.g., age, gender, Medicaid eligibility, income, etc.) and a diagnostic component (e.g., ICD-9 and/or ICD-10 codes).

In accordance with aspects of this disclosure, server 22 may apply analytical methods to automatically identity any patient information needing an update or verification of their conditions affecting HCC status. For instance, server 22 may push an alert to client computing device 12 when an HCC is within 45 days of expiration and needs to be updated. Server 22 may also implement aspects of this disclosure to enable a user to view the evidence within the documentation as to where the diagnosis code came from. Due to rules governing valid sources for determining HCCs (e.g. the diagnosis must come from a direct face-to-face evaluation of the patient by a physician), aspects of this disclosure may enable a user to view the evidence relating to diagnosis origination. Additionally, server 22 may apply analytics to compare a patient's current HCC status with the patient's HCC status from previous years to provide related statistics for individual patients as well as patient/member populations. By comparing a patient's HCC status to past data for the patient as well as patient groups, server 22 may implement the techniques described herein to provide potentially valuable and accurate information to the insurer, for use in management activities such as resource planning and contract negotiations.

A potential outcome of the disclosed techniques is a solution that integrates concurrent HCCs management into multiple key roles within a healthcare system. Example workflows are listed, and are described in greater detail below. In one example workflow, a physician or a care team member interaction with patients and associated documentation tasks is described. In another example, a workflow is described for CDI specialists who may assist physicians in identifying cases needing greater clarification or specificity regarding a patient's HCC status. Another example workflow is associated with community case managers who may use HCC data to manage the healthcare system's population affected by HCC reimbursement and risk plans. Another example workflow is associated with HIM coders, who may verify ICD-9 or ICD-10 diagnoses and related information in a medical record. In another example, a workflow is described for business management personnel and/or analysts who may use analytics for resource planning and contract negotiations. Another example workflow is associated with system analysts who provide HCC data to payers and Medicare as needed, as well as to hospital finance teams that can identify cases subject to internal audit for lack of documentation of existing chronic diseases.

FIG. 2 is a block diagram illustrating the server of the example of FIG. 1. As shown in FIG. 2, server 22 includes processor 21, one or more input devices 25, one or more output devices 26, communication interface 23, and memory 27. Server 22 may be a computing device configured to perform various tasks and interface with other devices, such as repository 24 and client computing devices (e.g., client computing device 12 of FIG. 1). Although repository 24 is shown external to server 22, server 22 may include repository 24 within a server housing in other examples. Server 22 may also include other components and modules related to the processes described herein and/or other processes. The illustrated components are shown as one example, but other examples may be consistent with various aspects described herein.

Processor 21 may include one or more general-purpose microprocessors, specially designed processors, application specific integrated circuits (ASIC), field programmable gate arrays (FPGA), a collection of discrete logic, and/or any type of processing device capable of executing the techniques described herein. In some examples, processor 21 or any other processors herein may be described as a computing device. In one example, memory 27 may be configured to store program instructions (e.g., software instructions) that are executed by processor 21 to carry out the techniques described herein. Processor 21 may also be configured to execute instructions stored by repository 24. Both memory 27 and repository 24 may be one or more storage devices. In other examples, the techniques described herein may be executed by specifically programmed circuitry of processor 21. Processor 21 may thus be configured to execute the techniques described herein. Processor 21, or any other processes herein, may include one or more processors.

Memory 27 may be configured to store information within server 22 during operation. Memory 27 may comprise a computer-readable storage medium. In some examples, memory 27 is a temporary memory, meaning that a primary purpose of memory 27 is not long-term storage. Memory 27, in some examples, may comprise as a volatile memory, meaning that memory 27 does not maintain stored contents when the computer is turned off. Examples of volatile memories include random access memories (RAM), dynamic random access memories (DRAM), static random access memories (SRAM), and other forms of volatile memories known in the art. In some examples, memory 27 is used to store program instructions for execution by processor 21. Memory 27, in one example, is used by software or applications running on server 22 to temporarily store information during program execution.

Input devices 25 may include one or more devices configured to accept user input and transform the user input into one or more electronic signals indicative of the received input. For example, input devices 25 may include one or more presence-sensitive devices (e.g., as part of a presence-sensitive screen), keypads, keyboards, pointing devices, joysticks, buttons, keys, motion detection sensors, cameras, microphones, or any other such devices. Input devices 25 may allow the user to provide input via a user interface.

Output devices 26 may include one or more devices configured to output information to a user or other device. For example, output device 54 may include a display screen for presenting visual information to a user that may or may not be a part of a presence-sensitive display. In other examples, output device 54 may include one or more different types of devices for presenting information to a user. Output devices 26 may include any number of visual (e.g., display devices, lights, etc.), audible (e.g., one or more speakers), and/or tactile feedback devices. In some examples, output devices 26 may represent both a display screen (e.g., a liquid crystal display or light emitting diode display) and a printer (e.g., a printing device or module for outputting instructions to a printing device). Processor 21 may present a user interface via one or more of input devices 25 and output devices 26, whereas a user may control the abstraction and/or coding of clinical documentation via the user interface. In some examples, the user interface generated and provided by server 22 may be displayed by a client computing device (e.g., client computing device 12).

Server 22 may utilize communication interface 23 to communicate with external devices via one or more networks, such as network 20 in FIG. 1, or other storage devices such as additional repositories over a network or direct connection. Communication interface 23 may be a network interface card, such as an Ethernet card, an optical transceiver, a radio frequency transceiver, or any other type of device that can send and receive information. Other examples of such communication interfaces may include Bluetooth, 4Q and WiFi radios in mobile computing devices as well as USB. In some examples, server 22 utilizes communication interface 23 to wirelessly communicate with external devices (e.g., client computing device 12) such as a mobile computing device, mobile phone, workstation, server, or other networked computing device. As described herein, communication interface 23 may be configured to receive clinical documentation, codes, and/or transmit suggested codes and/or queries over network 20 as instructed by processor 21.

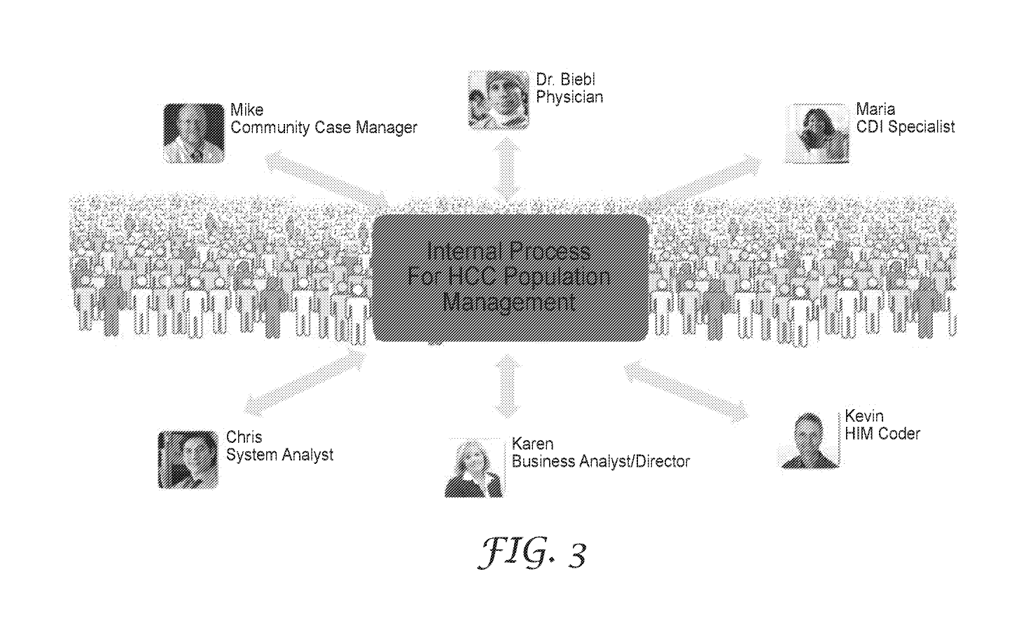

FIG. 3 is a conceptual diagram illustrating a general schematic associated with the techniques of this disclosure. System 28 illustrates various entities that may collaborate using aspects of this disclosure, including physicians, CDI specialists, HIM coders, system analysis, community case managers, and business analysts.

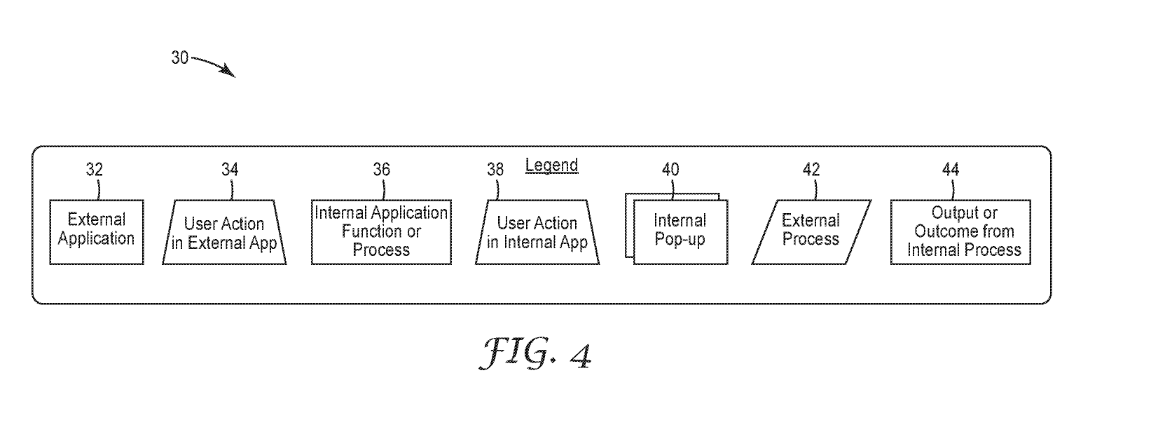

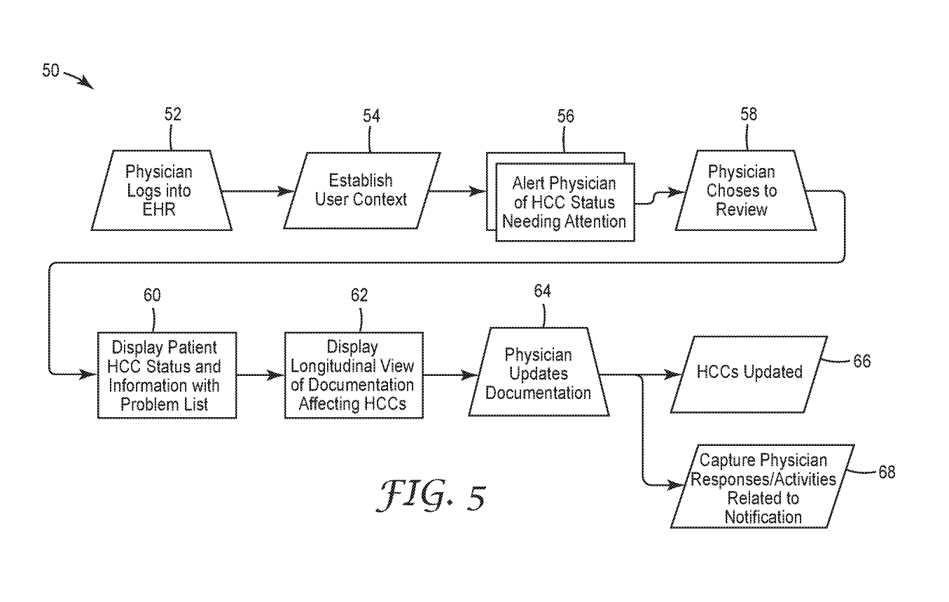

FIG. 4 is a block diagram that illustrates a legend 30 for various flowcharts in the accompanying drawings. Various components illustrated in FIG. 4 are used to illustrate steps in the flowcharts described below with respect to FIGS. 5, 7, and 9-12. Legend 30 includes representations of an external application 32, a user action 34 in an external application, an internal application function/process 36, a user action 38 in an internal application, an internal pop-up 40, an external process 42, and an output/outcome 44 from an internal process. External application 32, internal application function/process 36, and output/outcome 44 from an internal process are represented using rectangles of different dimensionalities in legend 30. In the particular example of FIG. 4, internal application function/process 36 is illustrated using a wider rectangle than external application 32, and output/outcome 44 from an internal process is represented by a wider rectangle than both internal application function/process 36 and external application 32.

User actions 34 and 38 (in an external application and internal application, respectively) are illustrated using trapeziums, or trapezoid outlines. Internal pop-up (alternatively, “popup”) 40 is illustrated using cascaded rectangles. External process 42 is illustrated using a parallelogram. Legend 30 maps shapes to types of steps, as used in FIGS. 5, 7, and 9-12 to categorize the individual steps of the flowcharts into various types. While the steps of the various flowcharts described below are described with respect to components of system 10 of FIG. 1 for purposes of illustration, it will be appreciated that various other devices may implement the techniques of this disclosure, as well.

FIG. 5 is a flowchart illustrating an example process or workflow 50 by which physician or a care team member interacts with patients using one or more systems and/or techniques of this disclosure. Documentation associated with the physician's or care team member's interaction with the patient is also illustrated and described in workflow 50. According to workflow 50, a physician is notified of a patient's HCC status when the physician logs into an electronic health record (EHR) system, and views a patient record. Techniques of this disclosure enable server 22 and client computing device 12 to synchronize (e.g., “synch” or “sync”) with the EHR environment, so that the user and patient context is shared between the EHR and the computing environment provided by client computing device 12. The physician may then use client computing device 12 to review relevant HCC information, or explore the evidence for each HCC from either the current encounter or previous encounters or claims. In some instances, the physician can provide further documentation via client computing device 12 to verify or update the HCC status. Physician responses and related activities are captured (e.g., by NLP engine 14) for additional statistical capabilities, such as physician staff management.

Workflow 50 may begin when a physician logs into an EHR using client computing device 12 (52). While described as being performed by a physician as an example, it will be appreciated that client computing device 12 may enable various users, such as a care team member, to implement step 52. In turn, one or both of server 22 and/or client computing device 12 may establish a user context for the EHR login (54). Additionally, server 22 and/or client computing device 12 may alert the physician of an HCC status needing attention (56). For example, server 22 may detect a condition (e.g., data discrepancy), and in response, may push an alert to client computing device 12. In turn, client computing device 12 may output the alert to a user, via one or more output devices (e.g., a monitor, one or more speakers, etc.)

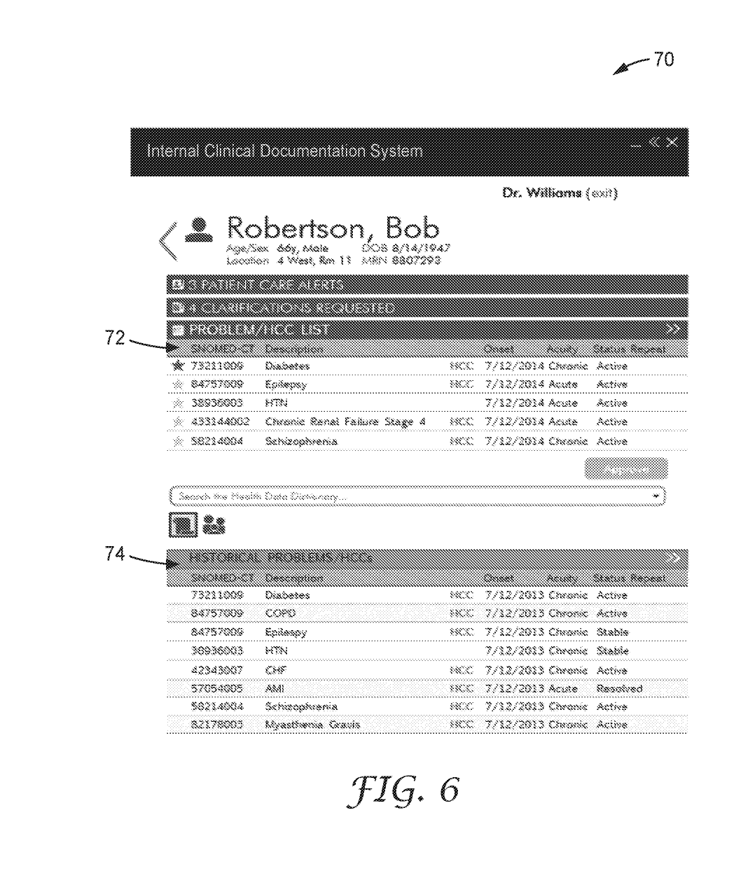

Client computing device 12 may receive an input indicating that a user (e.g., a physician) has chosen to review the data that instigated the alert (58). In turn, client computing device 12 may display the patient's HCC status and other related information with a problem list (60). An example problem list is illustrated in FIG. 6 and described in greater detail below. In turn, client computing device 12 may display a longitudinal view of documentation that affects the HCCs (62). An example of a longitudinal view of documentation (such as a longitudinal list of diagnoses) is illustrated in FIG. 6 and described in greater detail below.

Client computing device 12 may receive a user input indicating that a physician (or other user) updates the documentation displayed via the problem list and/or the longitudinal view of the documentation (62). In various examples, client computing device 12 may communicate the information update(s) to server 22. In turn, server 22 may update the HCCs pertaining to the patient, based on the update(s) received from client computing device 12 (66).

FIG. 6 is a screenshot illustrating a user interface (UI) 70 through which physicians can view the current and historical HCC status of a patient, using client computing device 12. Additionally, by augmenting problem list 72, (e.g., a feature in the 3M® 360 Encompass MD solution), the physician may use client computing device 12 to update data included in the diagnostic component of HCC information according to aspects of this disclosure. As shown in FIG. 6, diagnoses of problem list 72 that map to a corresponding HCC are marked “HCC.” Additionally, in the specific example of FIG. 6, longitudinal diagnosis list 74 shows three diagnoses from the previous year that may or may not need to be updated, based on the physician's evaluation. Longitudinal diagnosis list 74 may, in some instances, represent a historical diagnosis list for the patient, such as a listing of ICD-coded diagnoses that were recorded for the patient in one or more prior years.

In the particular example of longitudinal diagnosis list 74, if the previous congestive heart failure diagnosis (CHF) is still current, the physician could interact with client computing device 12 via UI 70, to indicate that the CHF condition is still current. For instance, the physician may select the CHF condition to indicate that, based on the still-current status of the CHF condition in longitudinal diagnosis list 74 and other factors from a physical evaluation, that the patient's overall HCC scoring would likely increase.

Server 22 may implement the techniques of this disclosure to populate longitudinal diagnosis list 74 with certain ICD codes that the physician had selected as being current in the immediately previous calendar year. For instance, server 22 may determine that the physician had selected an ICD code based on a physical exam of the patient during the prior calendar year, but that the physician has not selected the ICD code for the current year. While described with respect to comparing current ICD codes to ICD codes from the immediately preceding calendar year for purposes of discussion, it will be appreciated that, in various implementations, server 22 may also determine possible errors by comparing the current ICD-9 codes to ICD-9 codes that were entered for multiple preceding calendar years (e.g., a two-year window, a three-year window, etc.).

Using the techniques described herein, server 22 may determine that the omission of the particular ICD code is a possible error, and may push an alert to client computing device 12 indicating the possible error. In turn, client computing device 12 may add the corresponding condition to longitudinal diagnosis list 74, to prompt the physician to assess and address the condition for the current year. In this manner, server device 22 and client device 12 may implement the techniques of this disclosure to detect possible errors in ICD-9 code entries, and prompt the physician to correct the errors before the errors affect insurance-related determinations. Thus, the systems and techniques of this disclosure provide the potential technical benefit of identifying data errors and providing recovery options in case of data errors occurring.

While described with respect to physician use, client computing device 12 may also generate UI 70 in cases where the user is a clinician or a HIM coder. Additionally, server 22 may generate prompts differently for different users. For instance, if the user of client computing device 12 is a physician (determined by login information, IP address, etc.), then server 22 may implement a granular approach to selecting ICD codes to flag as possible errors. As one example, for a physician end-user, server 22 may only select omitted ICD codes from a pool of codes that fall within the top 20% (or even just the top 10%) of conditions. However, if the end user is a clinician, server 22 may flag possible errors for missing ICD codes that are selected from a broader pool. If the end user is a HIM coder, server 22 may flag possible errors for missing ICD codes that are selected from a still broader pool.

In this manner, server 22 may implement the techniques of this disclosure to customize error flagging for different categories of end users. For instance, the techniques may enable the clinician to double check errors of mid-level severity that server 22 did not flag for the physician. Along the same lines, the techniques may enable a HIM coder to check for lower severity errors that server 22 did not flag for either the physician or the clinician. In this manner, server 22 may implement the techniques of this disclosure to subject different categories of errors to varying levels of scrutiny, thereby implementing a beginning-to-end error flagging mechanism in terms of the medical evaluation process. Thus, the systems and techniques of this disclosure provide potential technical improvements, such as by introducing enhanced error trapping and mechanisms for error resilience.

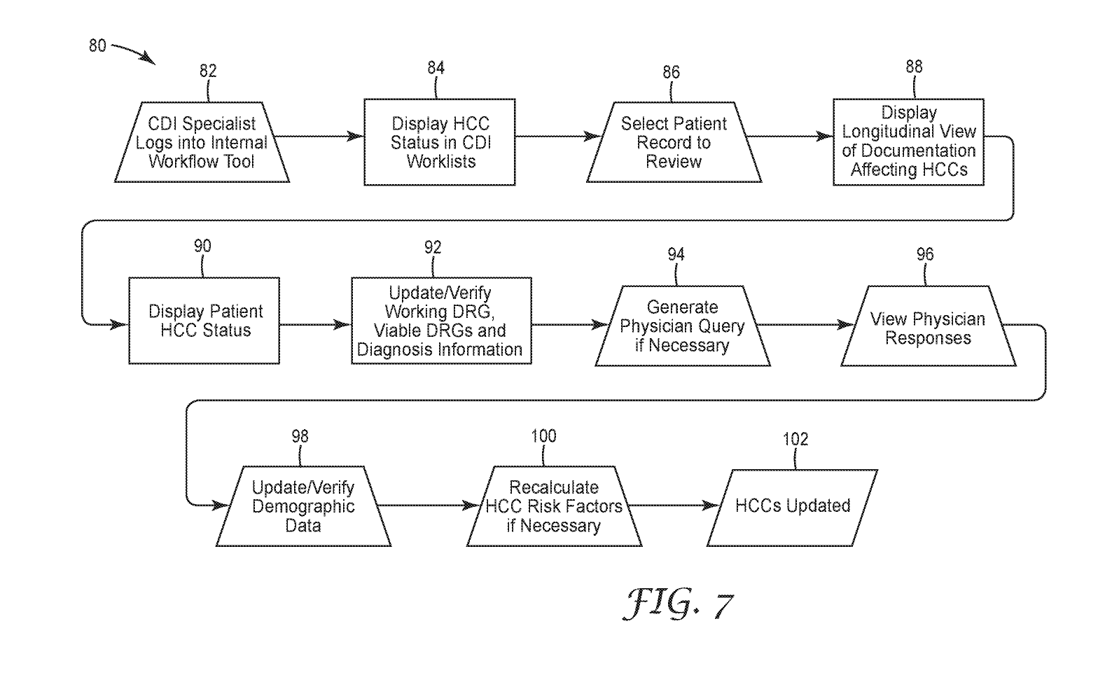

FIG. 7 is a flowchart illustrating workflow 80 associated with clinical documentation improvement (CDI) specialists' interaction with tools of this disclosure. CDI specialists may work concurrently with physicians and care team members in identifying opportunities to clarify conditions and diagnoses (expressed using ICD codes) related to HCC assignment. As shown in the example of workflow 80, techniques of this disclosure may enable a CDI specialist to select a patient record for review, and view a longitudinal list (e.g., longitudinal diagnosis list 74 of FIG. 6) of HCC-affecting diagnosis codes. Additionally, the CDI specialist(s) may view the current HCC score for the patient, and may make any necessary updates to the HCC-affecting diagnoses. In the example of workflow 80, the CDI specialist(s) may also submit any updates for physician feedback or approval. Pending physician feedback and approval, the CDI specialist(s) may update one or both components (demographic and/or diagnostic) components of the HCC-affecting data, and spur an HCC level update for the patient. By bringing the error checking process closer to the point of care (e.g., by enabling CDI specialists to work with physicians to correct possible errors), server 22 and client computing device 12 may enhance accuracy and completeness of the patient data provided to insurers.

Workflow 80 may begin when client computing device 12 receives a login from a user (e.g., a CDI specialist) via an internal workflow tool (82). In turn, client computing device 12 may display the HCC status in one or more CDI worklists (84). Client computing device 12 may receive input selecting a patient record for review (86). For instance, the CDI specialist may select the patient record for review via UI 70 illustrated in FIG. 6 and described above.

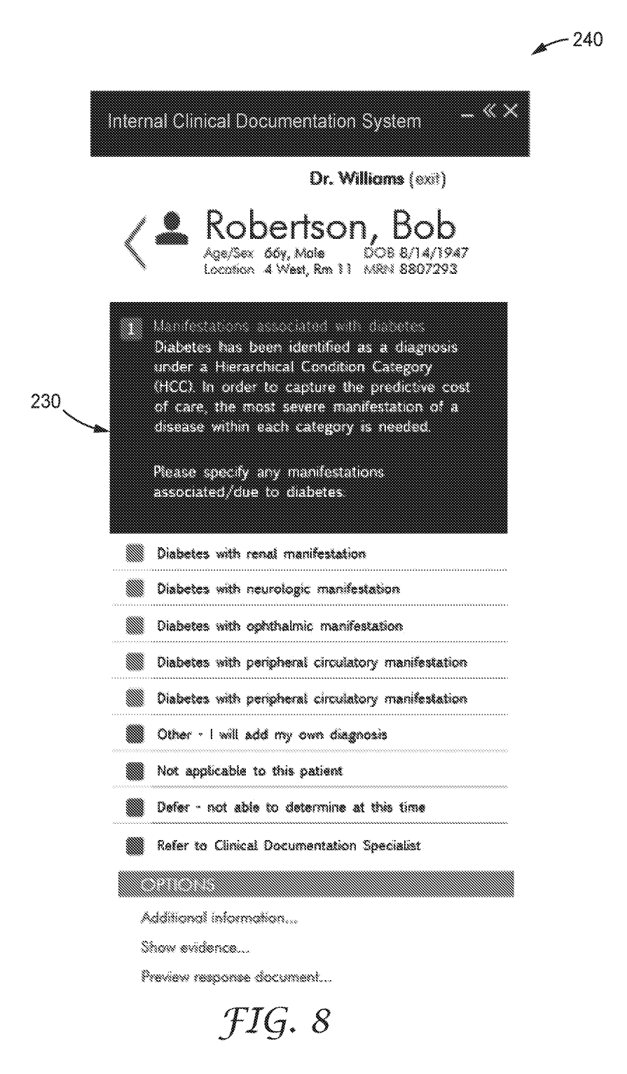

In turn, client computing device 12 may display the patient's HCC status (90). An example of a patient HCC status display is illustrated in FIG. 8, and described in further detail below. Client computing device 12 may update and/or verify one or more of working DRG(s), viable DRG(s), and diagnosis information (92). For instance, client computing device 12 may update one or more of the records listed above by relaying change information to server 22. Additionally, server 22 and/or client computing device 12 may generate a physician query (94). It will be appreciated that the generation of a physician query is an optional step, and that client computing device 12/server 22 may only generate the physician query if necessary, such as in scenarios where the received update information requires physician approval or inspection. In turn, client computing device 12 may output any physician response(s) for display, thereby enabling the CDI specialist to view the physician response(s) (96). Again, it will be appreciated that the output of any physician response is an optional step, contingent upon whether or not any physician response was received at all. In turn, computing device 12 may cause server 22 to update and/or verify demographic data based on the received input(s) and any physician response(s) that may have been received (98).

Based on the update information and any received physician responses, server 22 may recalculate HCC risk factors, if a recalculation is necessary (100). For instance, server 22 may determine whether any recalculation is necessary at all, based on the nature of the update information and any potential physician response(s). In turn, server 22 may update the HCCs based on any such recalculation (102).

FIG. 8 is a screenshot 90 illustrating a query 92 to the physician originating from the CDI specialist. In the example of screenshot 90, query 92 reflects an inquiry from the CDI specialist to a physician. By way of query 92, the CDI specialist is asking the physician whether additional, more complex healthcare conditions are manifestations of the patient's diabetes.

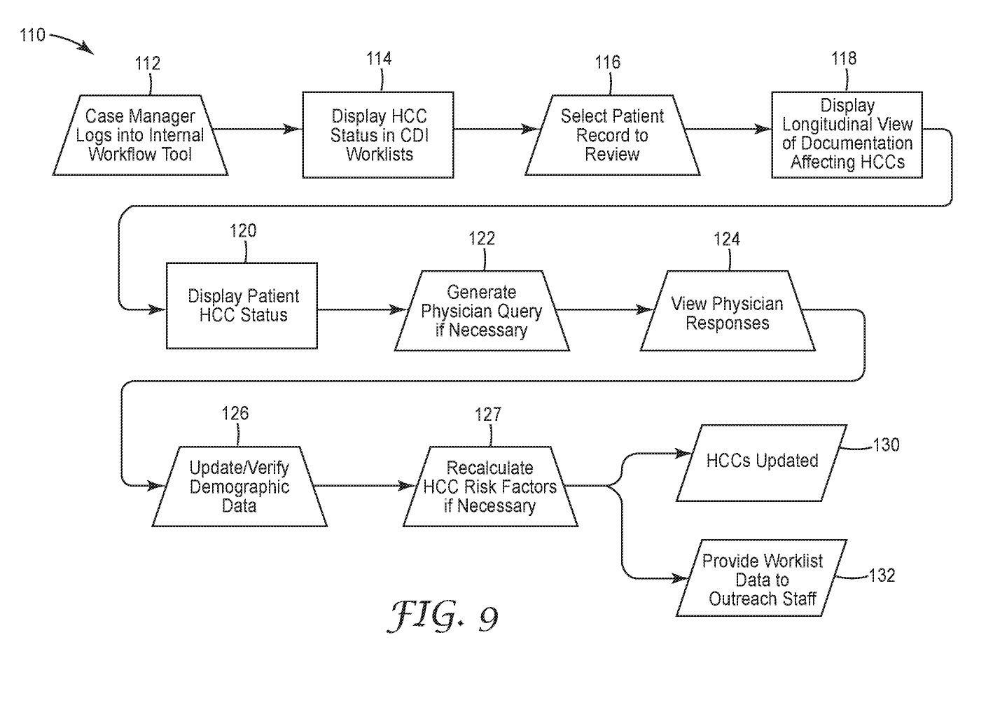

FIG. 9 is a flowchart illustrating workflow 110 associated with a community case manager's role in the medical reporting process. According to workflow 110, a community case manager may enter ICD codes using client communication device 12. Additionally, server 22 may flag possible errors to the community case manager according to the techniques described herein. A community case manager may fill in any gaps for members (patients) who are not currently receiving care. The community case manager will have access to lists of members whose HCC status need to be updated, especially those members/patients with a history of greater HCC risk scores.

As shown in the example of workflow 110, techniques of this disclosure may enable a community case manager to select a patient record for review, and view a longitudinal list (e.g., longitudinal diagnosis list 74 of FIG. 6) of HCC-affecting diagnosis codes. Additionally, the community case manager may view the current HCC score for the patient, and may make any necessary updates to the HCC-affecting diagnoses. In the example of workflow 110, the community case manager may also submit any updates for physician feedback or approval. Pending physician feedback and approval, the community case manager may update one or both components (demographic and/or diagnostic) components of the HCC-affecting data, and spur an HCC level update for the patient. As shown in the example of workflow 110, the community case manager may also provide worklist data pertaining to the patient to outreach staff.

Workflow 110 may begin when client computing device 12 receives a login via an internal workflow tool, such as a login by a case manager (112). In turn, client computing device may output the HCC status for display, such as in one or more CDI worklists (114). Client computing device 12 may receive input selecting a patient record for review (116). For instance, the case manager may select the patient record for review via UI 240 illustrated in FIG. 8 and described above.

In turn, client computing device 12 may display the patient's HCC status (120). An example of a patient HCC status display is illustrated in FIG. 8, and described in further detail above with respect to FIG. 8. Server 22 and/or client computing device 12 may generate a physician query (122). It will be appreciated that the generation of a physician query is an optional step, and that client computing device 12/server 22 may only generate the physician query if necessary, such as in scenarios where the received update information requires physician approval or inspection. In turn, client computing device 12 may output any physician response(s) for display, thereby enabling the CDI specialist to view the physician response(s) (124). It will be appreciated that the output of any physician response is an optional step, contingent upon whether or not any physician response was received at all. In turn, client computing device 12 may cause server 22 to update and/or verify demographic data based on the received input(s) and any physician response(s) that may have been received (126).

Based on the update information and any received physician responses, server 22 may recalculate HCC risk factors, if a recalculation is necessary (127). For instance, server 22 may determine whether any recalculation is necessary at all, based on the nature of the update information and any potential physician response(s). In turn, server 22 may update the HCCs based on any such recalculation (130). Additionally (e.g., in parallel with step 130), client computing device 12 may provide worklist data to outreach staff (132).

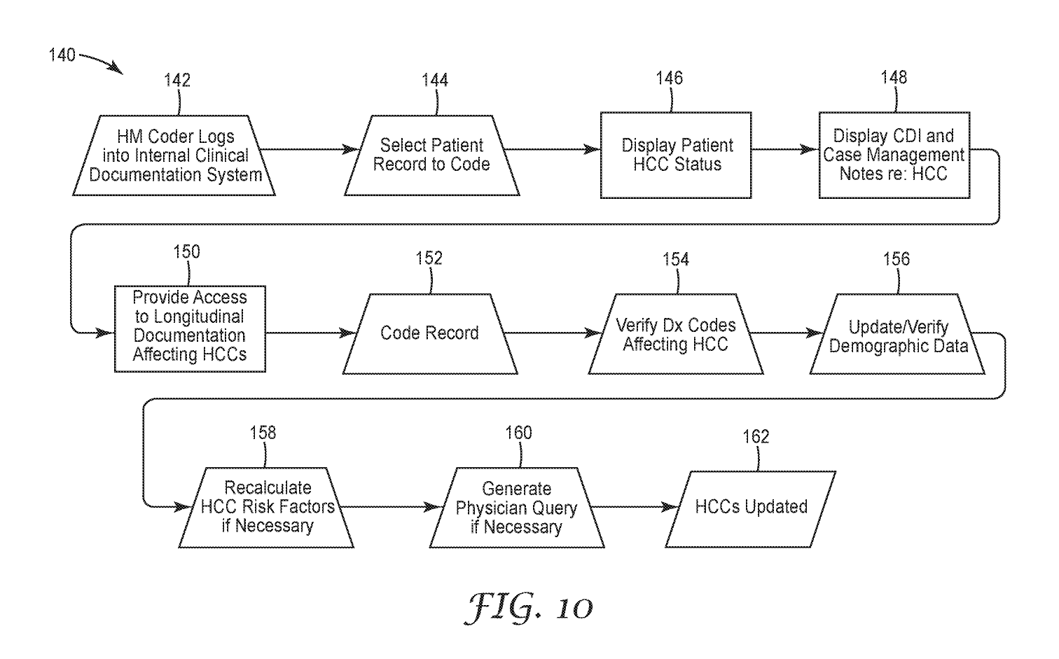

FIG. 10 is a flowchart illustrating workflow 140 associated with the role of HIM coders, who may verify ICD-9 or ICD-10 diagnoses and related information in a medical record. The role of a HIM coder role has a bearing on the accuracy of HCCs determined from diagnosis codes. Ultimately, the output of the coding process determines which HCCs, if any, can be assigned to the patient based on the diagnoses. According to the techniques of this disclosure, server 22 may flag potential errors selected from a broad range of diagnoses to HIM coders. Additionally, HIM coders have access to the activity of the CDI specialists, community case managers, and physicians involved in the patients care. Coders may also have access to the patient's longitudinal record to examine documentation from previous encounters in verifying a patient's current HCC status and risk scoring. Thus, based on the HIM coders' broad-ranging access to diagnostic information, server 22 may implement the techniques of this disclosure to select possible errors from a broad range of ICD codes, when flagging possible errors to a HIM coder end user of client computing device 12.

As shown in the example of workflow 140, techniques of this disclosure may enable a HIM coder to and view notes entered by a CDI specialist and/or a community case manager. The HIM coder may also view a longitudinal list (e.g., longitudinal diagnosis list 74 of FIG. 6) of HCC-affecting diagnosis codes. In turn, the HIM coder may code the patient record, such as by entering ICD codes for any diagnoses included in the record. The HIM coder may also verify the HCC-affecting diagnosis codes. HIM coders may also verify and (where necessary) update the demographic component of the HCC data, as shown in workflow 110. In the example of workflow 140, the HIM coder may recalculate HCC scores for the patient, and may generate a query for the physician in cases of potential errors or omissions.

Workflow 140 may begin when client computing device 12 receives a login via an internal clinical documentation system, such as a login by a HM coder (142). In turn, client computing device 12 may receive input selecting a patient record to code (144). Additionally, client computing device 12 may display the patient's HCC status (146). An example of a patient HCC status display is illustrated in FIG. 8, and described in further detail above with respect to FIG. 8.

In turn, client computing device 12 may output, for display, CDI and/or case management notes pertaining to the HCC status (148). Additionally, client computing device 12 may provide access to longitudinal documentation affecting the HCCs (150). For instance, client computing device 12 may query server 22 for the HCC-related data, and provide access to the longitudinal documentation affecting the HCCs via a UI. Server 22 and/or client computing device 12 may code the record, such as by using input received from the HM coder (152). Additionally, server 22 and/or client computing device 12 may verify the diagnosis (or “Dx”) codes affecting the HCC (154). In turn, client computing device 12 may cause server 22 to update and/or verify demographic data (156).

Based on the updates and/or verification information, server 22 may recalculate HCC risk factors, if a recalculation is necessary (158). For instance, server 22 may determine whether any recalculation is necessary at all, based on the nature of the update information and any potential physician response(s). Additionally, server 22 and/or client computing device 12 may generate a physician query (160). It will be appreciated that the generation of a physician query is an optional step, and that client computing device 12/server 22 may only generate the physician query if necessary, such as in scenarios where the received update information requires physician approval or inspection. In turn, server 22 may update the HCCs based on any recalculation(s) (162).

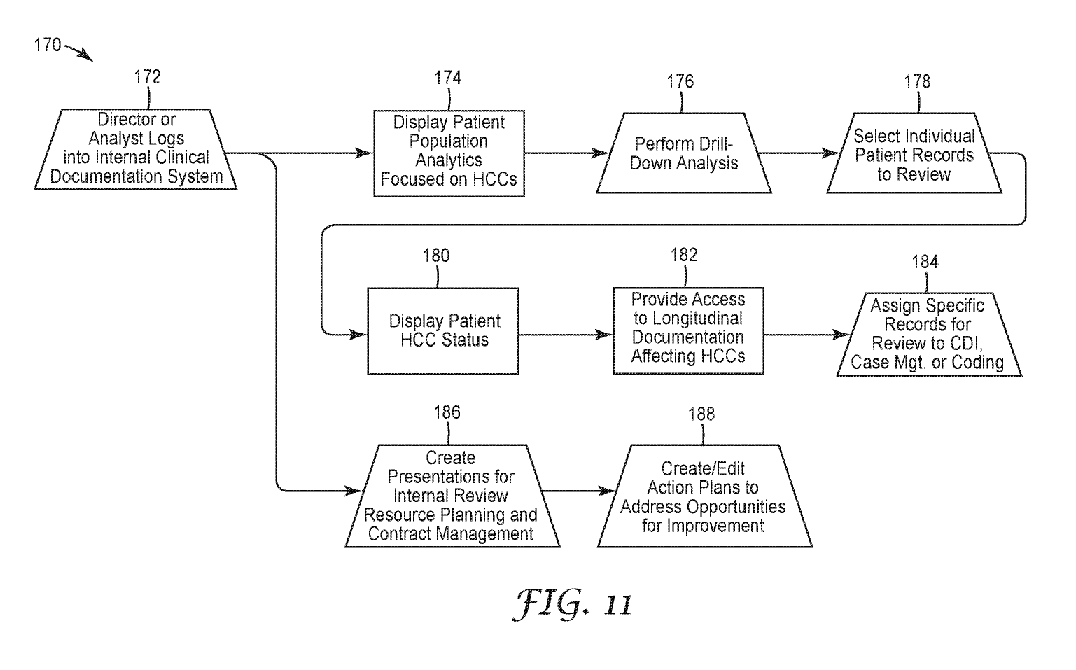

FIG. 11 is a flowchart illustrating workflow 170 for business management personnel and/or analysts who may use analytics for resource planning and contract negotiations. According to the techniques of this disclosure, managers and business analysts will have concurrent data from which to determine the organization's HCC status as it relates to a) population management, b) financial risk c) resource planning, and d) contract negotiation. Thus, the analytics provided by the techniques of this disclosure may help the business management personnel identify gaps or trends based on HCC coding. For instance, the business management personnel may predict risk score movements among the population and use the predictive data to negotiate new contracts with healthcare providers and/or payers, such as CMS. In this way, various error-detection aspects of this disclosure provide the technical benefit of systems that are better equipped to trap data errors, to enable greater error resilience, and to mitigate potential propagation of data errors.

Workflow 170 may begin when client computing device 12 receives a login via an internal clinical documentation system, such as a login by a director or analyst (172). In turn, client computing device 12 may display patient population analytics focused on HCCs (174). Additionally, (e.g., in parallel with step 174), client computing device 12 may create one or more presentations for internal review resource planning and contract management (186). It will be appreciated client computing device 12 may perform steps 174 and 186 in parallel, or at partially-overlapping timeframes, or independently of each other. Upon creating presentations for internal review resource planning and contract management at step 186, client computing device 12 may create and/or edit one or more action plans to address opportunities for improvement (188).

Upon displaying the patient population analytics at step 174, client computing device 12 may perform (or cause server 22 to perform) a drill-down analysis (176). Client computing device 12 may receive input selecting one or more individual patient records for review (178). For instance, the case manager may select the patient record(s) for review via UI 240 illustrated in FIG. 8 and described above. In turn, client computing device 12 may display the patient HCC status (180).

Additionally, client computing device 12 may provide access to longitudinal documentation affecting the HCCs (182). For instance, client computing device 12 may query server 22 for the HCC-related data, and provide access to the longitudinal documentation affecting the HCCs via a UI. Server 22 may assign one or more specific records for review to CDI, case management, or coding (184).

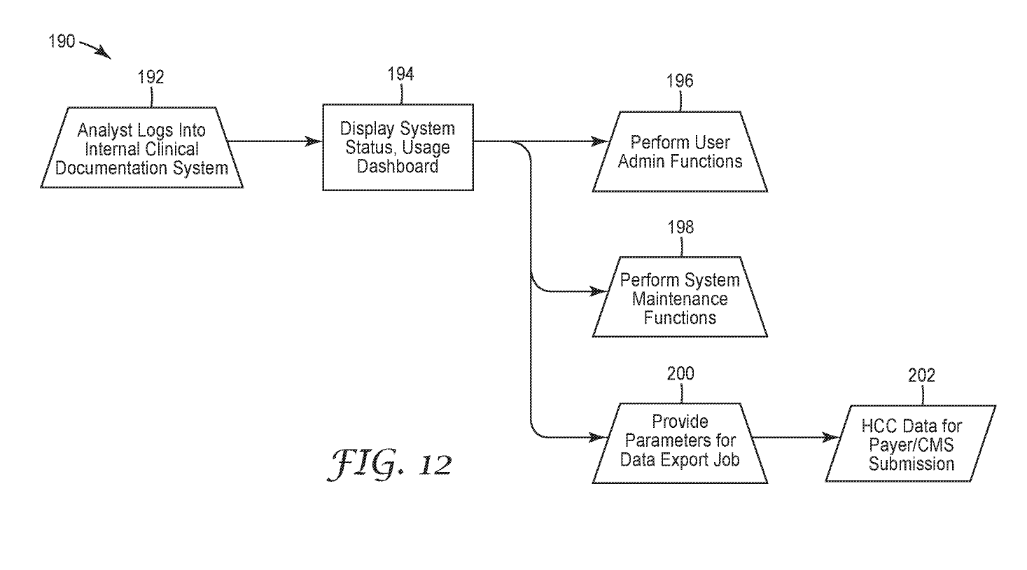

FIG. 12 is a flowchart illustrating workflow 130 associated with system analysts who provide HCC data to payers and Medicare as needed, as well as to hospital finance teams that can identify cases subject to internal audit for lack of documentation of existing chronic diseases. Healthcare organizations are required to submit data to payers and to CMS. Techniques of this disclosure may enable system administrators to fulfill data submission requirements as well as generate a patient list revealing lower risk scores of patients that may require internal audit to assess missing information. For instance, by flagging possible omissions of ICD codes for a patient, server 22 may alert system analyst end users of client computing device 12. If a system analyst recognizes a true error in the pushed alerts, the system analyst may rectify the error before the diagnostic information is made available to CMS.

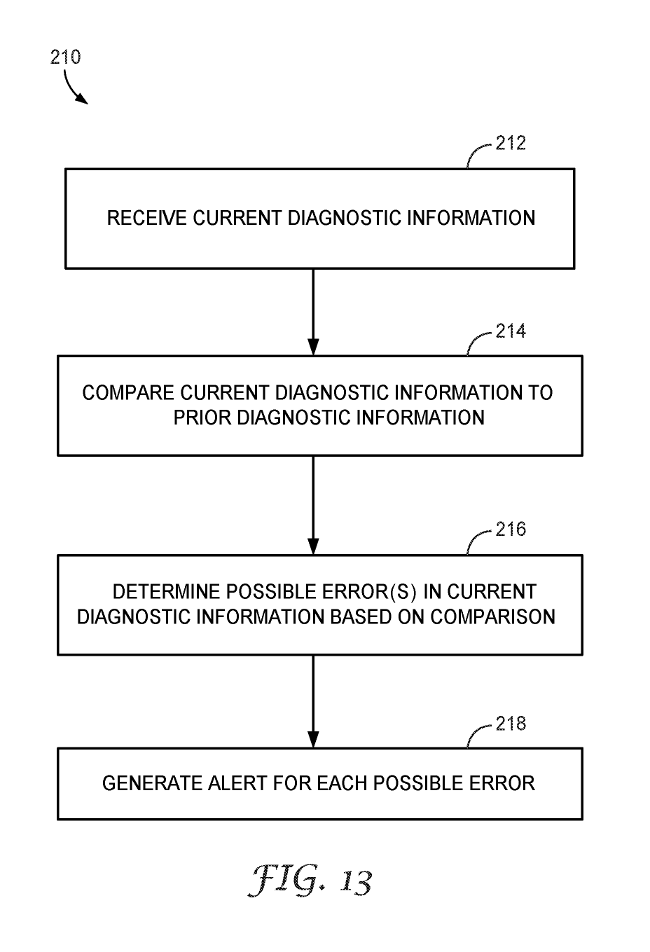

FIG. 13 is a flowchart illustrating an example process 140 that a computing device may perform to implement one or more of the medical information management and updating techniques of this disclosure. It will be appreciated that process 140 may be performed by a variety of devices. However, for purposes of discussion, process 140 is described herein as being performed by server 22 illustrated in FIGS. 1 and 2. Process 140 may begin when server 22 receives current diagnostic information for a patient. For instance, server 22 may receive the current diagnostic information over network 20 from client computing device 12. Additionally, server 22 may compare the current diagnostic information for the patient to prior diagnostic information for the patient. For instance, server 22 may compare ICD codes indicating the current diagnostic information (e.g., for the current calendar year) to ICD codes recorded for the same patient in the immediately preceding calendar year.

Server 22 may determine one or more possible errors in the current diagnostic information based on the comparison. For instance, server 22 may determine that an ICD code from the prior diagnostic information is missing from the current diagnostic information. Based on the particular diagnosis to which the omitted diagnostic code is mapped, server 22 may detect that the omission is a possible error in the current diagnostic information. In response to detecting one or more possible errors, server 22 may generate a corresponding alert for each possible error detected. For instance, server 22 may transmit each generated alert to client computing device 12 over network 20.

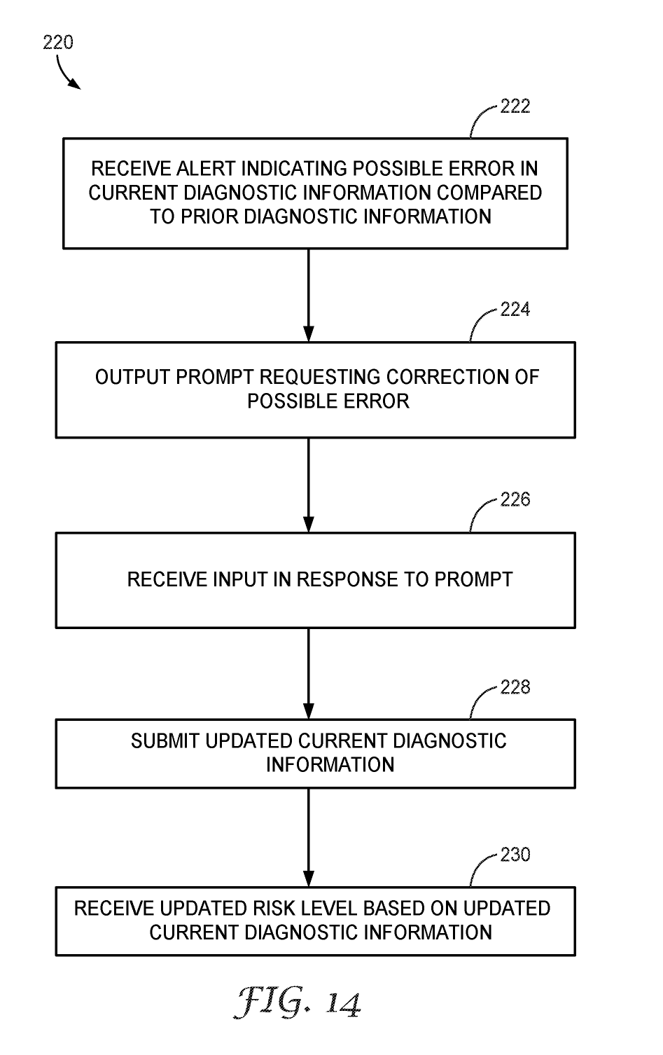

FIG. 14 is a flowchart illustrating an example process 160 that a computing device may perform to implement one or more of the medical information management and updating techniques of this disclosure. It will be appreciated that process 160 may be performed by a variety of devices. However, for purposes of discussion, process 140 is described herein as being performed by client computing device 12 illustrated in FIG. 1. Client computing device 12 may receive an alert indicating a possible error in current diagnostic information as compared to prior diagnostic information. For instance, client computing device 12 may receive the alert over network 20, from server 22.

Client computing device 12 may output a prompt requesting correction of the possible error. For instance, client computing device 12 may output the prompt to one or more medical staff members, such as a physician, a clinician, or a HIM coder. In turn, client computing device 12 may receive an input in response to the prompt. For instance, the medical staff member using client computing device 12 may provide an input that indicates a correction to the possible error, or an input that confirms that the previously recorded diagnostic information is correct. Using the input, client computing device 12 may update the current diagnostic information, either by correcting the flagged error, or by maintaining the current diagnostic information and clearing the corresponding error alert.

Client computing device 12 may submit the updated current diagnostic information to a health management system. For instance, client computing device 12 may transmit the updated current diagnostic information to server 22, over network 20. In turn, client computing device 12 may receive an updated risk level (e.g., HCC) for the patient, based on the updated current diagnostic information that was submitted.

The techniques of this disclosure may be implemented in a wide variety of computer devices, such as one or more servers, laptop computers, desktop computers, notebook computers, tablet computers, hand-held computers, smart phones, or any combination thereof. Any components, modules or units have been described to emphasize functional aspects and do not necessarily require realization by one or more different hardware units.

The disclosure contemplates computer-readable storage media comprising instructions to cause a processor to perform any of the functions and techniques described herein. The computer-readable storage media may take the example form of any volatile, non-volatile, magnetic, optical, or electrical media, such as a RAM, ROM, NVRAM, EEPROM, or flash memory that is tangible. The computer-readable storage media may be referred to as non-transitory. A server, client computing device, or any other computing device may also contain a more portable removable memory type to enable easy data transfer or offline data analysis.

The techniques described in this disclosure, including those attributed to server 22, repository 24, and/or computing device 100, and various constituent components, may be implemented, at least in part, in hardware, software, firmware or any combination thereof. For example, various aspects of the techniques may be implemented within one or more processors, including one or more microprocessors, DSPs, ASICs, FPGAs, or any other equivalent integrated or discrete logic circuitry, as well as any combinations of such components, remote servers, remote client devices, or other devices. The term “processor” or “processing circuitry” may generally refer to any of the foregoing logic circuitry, alone or in combination with other logic circuitry, or any other equivalent circuitry.

In various examples, the techniques described in this disclosure, including those attributed to server 22, repository 24, and/or computing device 100, and various constituent components, may be described as being implemented by a hardware control unit. As used herein, a hardware control unit may, for example, include or otherwise implement any combination of one or more processors, one or more field programmable gate arrays (FPGAs), one or more application specific integrated circuits (ASICs), and one or more application specific standard products (ASSPs). The hardware control unit may also comprise memory, both static (e.g., hard drives or magnetic drives, optical drives, FLASH memory, EPROM, EEPROM, etc.) and dynamic (e.g., RAM, DRAM, SRAM, etc.), or any other non-transitory computer readable storage medium capable of storing instructions that cause the one or more processors to perform the efficient medical information management techniques described in this disclosure. Thus, a hardware control unit in accordance with this disclosure may represent hardware or a combination of hardware and software to support the below described components, modules or elements, and the techniques should not be strictly limited to any particular embodiment described herein.

Such hardware, software, firmware may be implemented within the same device or within separate devices to support the various operations and functions described in this disclosure. For example, any of the techniques or processes described herein may be performed within one device or at least partially distributed amongst two or more devices, such as between server 22 and/or client computing device 12. In addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components.

The techniques described in this disclosure may also be embodied or encoded in an article of manufacture including a computer-readable storage medium encoded with instructions. Instructions embedded or encoded in an article of manufacture including a computer-readable storage medium encoded, may cause one or more programmable processors, or other processors, to implement one or more of the techniques described herein, such as when instructions included or encoded in the computer-readable storage medium are executed by the one or more processors. Example computer-readable storage media may include random access memory (RAM), read only memory (ROM), programmable read only memory (PROM), erasable programmable read only memory (EPROM), electronically erasable programmable read only memory (EEPROM), flash memory, a hard disk, a compact disc ROM (CD-ROM), a floppy disk, a cassette, magnetic media, optical media, or any other computer readable storage devices or tangible computer readable media. The computer-readable storage medium may also be referred to as storage devices.

In some examples, a computer-readable storage medium comprises non-transitory medium. The term “non-transitory” may indicate that the storage medium is not embodied in a carrier wave or a propagated signal. In certain examples, a non-transitory storage medium may store data that can, over time, change (e.g., in RAM or cache).

Various examples have been described herein. Any combination of the described operations or functions is contemplated. These and other examples are within the scope of the following claims.

1. A computer-implemented method for managing medical information, the method comprising:
receiving, by a computing device, current diagnostic information for a patient, the current diagnostic information including one or more current individual diagnoses for the patient;
comparing, by the computing device, the current diagnostic information to prior diagnostic information for the patient, the prior diagnostic information including one or more prior individual diagnoses for the patient;
determining, by the computing device, one or more possible errors in the current diagnostic information based on the comparison; and
generating, by the computing device, an alert associated with each possible error of the one or more possible errors,
detecting, by the computing device, co-morbidity information linking at least one current individual diagnosis and at least one prior individual diagnosis that does not correspond to any of the current individual diagnoses,
wherein generating the alert associated with at least one possible error of the one or more possible errors comprises generating the alert responsive to detecting the co-morbidity information.

2. The method of claim 1, wherein determining the one or more possible errors comprises:
determining, by the computing device, that at least one prior individual diagnosis for the patient does not correspond to any of the current individual diagnoses for the patient.

3. The method of claim 1, wherein comparing the current diagnostic information to the prior diagnostic information comprises:
selecting, by the computing device, a subset of the prior individual diagnoses to be compared to each of the current individual diagnoses.

4. The method of claim 3, wherein selecting the subset of the prior individual diagnoses comprises:
selecting, by the computing device the subset of the prior individual diagnoses based on a severity level of each of the prior individual diagnoses.

5. The method of claim 3, wherein selecting the subset of the prior individual diagnoses comprises:
selecting, by the computing device the subset of the prior individual diagnoses based on an end user designation.

6. The method of claim 5, wherein the end user designation comprises one of a physician, a clinician, or a health information management (HIM) coder.

7. (canceled)

8. The method of claim 1, further comprising:
determining, by the computing device, that at least one prior individual diagnosis that does not correspond to any of the current individual diagnoses affects a risk level assigned to the patient, wherein generating the alert associated with at least one possible error of the one or more possible errors comprises generating the alert responsive to determining that the risk level is affected.

9. The method of claim 8, wherein the current diagnostic information comprises an original version of the current diagnostic information, the method further comprising:
responsive to generating the alert, receiving, by the computing device, updated current diagnostic information for the patient, wherein the updated current diagnostic information includes one or more additional current individual diagnoses for the patient in comparison to the original version of the current diagnostic information; and
based on the updated current diagnostic information, updating, by the computing device, the risk level assigned to the patient to form an updated risk level assigned to the patient.

10. The method of claim 9, wherein the updated risk level assigned to the patient reflects one of: (i) an increased risk level associated with the patient, or (ii) a decreased risk level associated with the patient.

11. The method of claim 9, further comprising:
based on the updated risk level assigned to the patient, updating, by the computing device, funding information associated with the patient.

12-15. (canceled)

16. The method of claim 9, wherein updating the method further comprising:
based on the increased risk level, generating, by the computing device, an alert indicating one or more additional medical procedures associated with the one or more additional current individual diagnoses.

17. The method of claim 16, further comprising:
transmitting, by the computing device, the alert to a device accessible by one or more medical staff members associated with the patient.

18. The method of claim 17, wherein the one or more medical staff members associated with the patient include one or more of: (i) a physician attending the patient, (ii) one or more clinicians associated with the physician, or (iii) one or more health information management (HIM) coders associated with the physician and/or the clinician(s).

19. The method of claim 1, wherein the prior diagnostic information is associated with diagnostic information for the patient from only an immediately preceding calendar year.

20. The method of claim 1, wherein the prior diagnostic information is associated with diagnostic information for the patient from multiple preceding calendar years.

21. The method of claim 1, wherein each current individual diagnosis and each prior individual diagnosis is represented by a code specified in a standards document.

22. The method of claim 11, wherein the standards document comprises one of a ninth revision or a tenth revision of an International Classification of Diseases (ICD) standard.

23. The method of claim 1, wherein each current individual diagnosis and each prior individual diagnosis affects a risk level assigned to a patient, the risk level corresponding to a hierarchical condition category (HCC).

24. The method of claims 1, wherein the possible error comprises one or more of: (i) an omission, (ii) an erroneous code, or (iii) an erroneous keyword.

25. A computer-implemented method for managing medical information, the method comprising:
receiving, by a computing device, an alert indicating a possible error in current diagnostic information for a patient as compared to prior diagnostic information for the patient; and
outputting, by the computing device, a prompt requesting correction of the possible error, generating, by the computing device, the prompt such that prompt indicates that the at least one prior individual diagnosis does not correspond to any of the one or more current individual diagnoses included in the current diagnostic information,
receiving, by the computing device, an input in response to the prompt, the input including one of: (i) a confirmation that the at least one prior individual diagnosis does not correspond to any of the one or more current individual diagnoses included in the current diagnostic information, or (ii) a correction indicating an additional current individual diagnosis that corresponds to the at least one prior individual diagnosis,
wherein the alert indicating the possible error comprises an alert indicating that at least one prior individual diagnosis included in the prior diagnostic information does not correspond to any of one or more current individual diagnoses included in the current diagnostic information.

26-28. (canceled)

29. The method of claim 25, further comprising:
if the input comprises the correction indicating the additional current individual diagnosis, updating, by the computing device, the current diagnostic information based on the received input; and
submitting, by the computing device, the updated current diagnostic information via a clinical documentation system.

30. The method of claim 29, further comprising:
in response to submitting the updated current diagnostic information, receiving, by the computing device via the clinical documentation system, a risk level assigned with the patient.

31. The method of claim 30, wherein the received risk level comprises an increased risk level that is greater than an original risk level that was previously assigned to the patient.

32. The method of claim 31, wherein the increased risk level reflects an addition of the at least one additional current individual diagnosis to the current diagnostic information associated with the patient.

33. The method of claim 31, wherein each of the increased risk level and the original risk level maps to a predetermined hierarchical condition category (HCC).

34. The method of claim 25, wherein each current individual diagnosis and each prior individual diagnosis is represented by a code specified in a standards document.

35. The method of claim 34, wherein the standards document comprises one of a ninth revision or a tenth revision of an International Classification of Diseases (ICD) standard.

36. The method of claim 25, wherein each current individual diagnosis and each prior individual diagnosis affects a risk level assigned to a patient, the risk level corresponding to a hierarchical condition category (HCC).

37-43. (canceled)
